(19) United States
(12) Patent Application Publication
(10) Pub. No.: US 2025/0285704 A1
(43) Pub. Date: Sep. 11, 2025

(54) QUANTIFYING EFFECTS OF SEQUENCING VARIANCE ON CLASSIFICATION

(71) Applicant: TEMPUS AI, INC., Chicago, IL (US)

(72) Inventors: John L. Guittar, Chicago, IL (US); Stephane Guy R Wenric, New York, NY (US); Tushar A. Chandra, Chicago, IL (US)

(21) Appl. No.: 18/600,488

(22) Filed: Mar. 8, 2024

(51) Int. Cl. G16B 5/00; G16B 40/00

(52) U.S. Cl. CPC G16B 5/00; G16B 40/00

(57) ABSTRACT

A method includes receiving transcriptomic data; predicting standard deviation values for each gene expression values; for each sample: computing a simulated expression value, classifying the simulated expression values, computing confidence scores; and flagging the sample; and storing the confidence scores. A computing system includes a processor; and a memory having stored thereon instructions that when executed, cause the computing system to: receive transcriptomic data; predict standard deviation values for each gene expression values; for each sample: compute a simulated expression value, classify the simulated expression values, compute confidence scores; and flag the sample; and store the confidence scores. A computer-readable media includes non-transitory computer-readable instructions that, when executed, cause a computer to: receive transcriptomic data; predict standard deviation values for each gene expression values; for each sample: compute a simulated expression value, classify the simulated expression values, compute confidence scores; and flag the sample; and store the confidence scores.

### FIELD

The present disclosure is directed to methods and systems for quantifying effects of sequencing variance on classification, and more particularly, to techniques for performing role-based quality control checks on classification data.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Noise and artifacts are associated with gene expression levels due to the way gene expression levels are measured. For example, RNA expression quantification techniques (e.g., a sequencing machine, a microarray or another RNA expression quantification technique) introduce variance into the RNA-seq datasets that they generate. Specifically, sequencing a sample multiple times will produce multiple datasets, wherein a single gene common to the datasets can have different expression values in each respective dataset. For example, the multiple datasets may correspond to individual datasets generated by each run of the sample. Sometimes more than one sequencing run is required to obtain multiple datasets (one run can produce multiple datasets if two “copies”/aliquots/library preparations of the sample are run together on the sequencer, wherein each “copy” corresponds to a respective generated dataset).

This noise and artifact condition may lead to indeterminate results. For example, algorithms (e.g., a machine learning model, a classifier, etc.) that use continuous measures (e.g., gene expression values measured by next-generation sequencing RNA-seq) to perform a task (e.g., a classification-based patient binning/grouping task) may produce low-confidence or indeterminate results. In the classification example, expression levels of genes used in the algorithm may be within statistical margins of error of a number of classes (e.g., subtype A, subtype B, etc.). In such a case, a sample classified as subtype A may have RNA-seq values close to a threshold between two bins (classes), and if the sample were to be sequenced a second time, the variance introduced by the sequencer may cause the RNA-seq values to be different enough that the sample could be classified as subtype B.

This discordance and indeterminateness that is the result of sequencing noise and artifacts has been empirically observed, and is a problem in multiple contexts. First, indeterminate classification has been observed during validation of patient data that required multiple sequencing runs or reactions. This caused validation to fail, and required redesign of the testing. Second, indeterminate results have been observed in a clinical setting, preventing clinicians from selecting optimal therapy regimens. Communicating discordant results to patients due to a lack of reproducible sequence-based testing has the potential to erode patient confidence.

Accordingly, there is an opportunity for improved platforms and technologies for quantifying sequence variance on classification, to quantify effects of noise and artifacts on sequencing variance.

### SUMMARY

In an aspect, a computer-implemented quality control method for predicting whether a continuous value classifier will generate discordant classifications of a sample includes (i) receiving, via one or more processors, transcriptomic data including a plurality of observed gene expression values each corresponding to one of a plurality of tissue samples sequenced a plurality of times; (ii) generating, via a machine learning model, a predicted standard deviation value for each of the plurality of observed gene expression values; (iii) for each sample in the plurality of samples: (a) computing, for each simulation in a plurality of simulations, and for each of a plurality of genes, a respective simulated expression value, by drawing from a normal distribution based on an observed expression value for the gene and the predicted standard deviation value for the gene; (b) classifying, via one or more processors, the simulated expression values using the classifier, wherein classifying the simulated expression values includes counting a number of classifications; (c) computing, via one or more processors, a confidence score; and (d) when the confidence score is within a predetermined range, flagging the sample as a potentially discordant sample; and (iv) storing, in an electronic database, at least one of the confidence scores in association with the transcriptomic data.

In another aspect, a computing system includes one or more processors; and one or more memories, having stored thereon instructions that when executed, cause the computing system to: (i) receive, via the one or more processors, transcriptomic data including a plurality of observed gene expression values each corresponding to one of a plurality of tissue samples sequenced a plurality of times; (ii) generate, via a machine learning model, a predicted standard deviation value for each of the plurality of observed gene expression values; (iii) for each sample in the plurality of samples: (a) compute, for each simulation in a plurality of simulations, and for each of a plurality of genes, a respective simulated expression value, by drawing from a normal distribution based on an observed expression value for the gene and the predicted standard deviation value for the gene; (b) classify, via the one or more processors, the simulated expression values using the classifier, wherein classifying the simulated expression values includes counting a number of classifications; (c) compute, via the one or more processors, a confidence score; and (d) when the confidence score is within a predetermined range, flag the sample as a potentially discordant sample; and (iv) store, in an electronic database, at least one of the confidence scores in association with the transcriptomic data.

In yet another aspect, a computer-readable media includes a set of non-transitory computer-readable instructions that, when executed, cause a computer to: (i) receive, via the one or more processors, transcriptomic data including a plurality of observed gene expression values each corresponding to one of a plurality of tissue samples sequenced a plurality of times; (ii) generate, via a machine learning model, a predicted standard deviation value for each of the plurality of observed gene expression values; (iii) for each sample in the plurality of samples: (a) compute, for each simulation in a plurality of simulations, and for each of a plurality of genes, a respective simulated expression value, by drawing from a normal distribution based on an observed expression value for the gene and the predicted standard deviation value for the gene; (b) classify, via the one or more processors, the simulated expression values using the classifier, wherein classifying the simulated expression values includes counting a number of classifications; (c) compute, via the one or more processors, a confidence score; and (d) when the confidence score is within a predetermined range, flag the sample as a potentially discordant sample; and (iv) store, in an electronic database, at least one of the confidence scores in association with the transcriptomic data.

### BRIEF DESCRIPTION OF THE DRAWINGS

The figures described below depict various aspects of the system and methods disclosed herein. It should be understood that each figure depicts an example of aspects of the present systems and methods.

FIG. 1 depicts an exemplary computing system for quantifying sequencer variance, according to some aspects.

FIG. 2 depicts exemplary no-call language that may be included in a report comprising indeterminate results, according to some aspects.

FIG. 3 depicts an exemplary computer-implemented quality control method for predicting whether a continuous value classifier will generate discordant classifications of a sample, according to some aspects.

### DETAILED DESCRIPTION

### Overview

The present techniques are directed to methods and systems for quantifying sampling noise during RNA expression quantification, and effects on models such as classifiers.

Empirically, “flips” between patient classes/groups/bins (for example, where more than one RNA-sequencing dataset is associated with the same sample, and the datasets are not all classified into the same group or bin) have been observed during algorithm validation. These discordant classifier results can be confusing for the clinician treating the patient, especially if each patient group is associated with a distinct therapy recommendation. In one example, the cause of these classification flips have been narrowed down to noise and artifacts in data generated during next-generation sequencing. Thus, the present techniques, which enable quantifying and predicting such flips or discordant results, provide a significant improvement to sequence data-based prediction techniques, by notifying researchers/clinicians of such potential indeterminate results, and by performing automatic mitigation.

In clinical cases (for example, where a sequenced sample is associated with a patient or organoid and the classifier result is reported to a clinician, especially to aid the clinician in selecting a therapy for a patient), it is useful to know when a sample might produce a discordant result on re-run (i.e., an indeterminate result), so that the clinician can receive accurate information to best select a therapy regimen for the patient, especially if subtype A is matched with therapy A, and subtype B is matched with therapy B. It is useful to flag these indeterminate samples when reporting results to individual patients or clinicians to ensure a high degree of patient-level reproducibility. Of course, the subtypes A and B are mere examples (any number of classes may be chosen, as in a multi-stage model).

The present techniques may use proprietary estimates of confidence in gene expression values (e.g., for the probe enhanced whole-exome capture RNA-seq platform) to predict the likelihood of a sample producing discordant subtype results upon a re-run. Herein, samples with a high likelihood of showing discordant results upon multiple re-runs are considered low-confidence or indeterminate samples. In one example, samples that fell below an 85% confidence threshold may be classified as low-confidence and reported to patients (via their clinicians) as “no-calls”. In another example, those samples may be reported as indeterminate, equivocal, etc. Conversely, samples with confidence scores greater than or equal to 85% may be classified as high confidence and thus reportable to patients/clinicians. In other embodiments, the threshold for reporting may be 60%, 65%, 70%, 75%, 80%, 90%, 95%, etc.

The present techniques may include a simulation-based approach to calculate subtype confidence scores for each sample based on its proximity to the threshold between two subtypes. In some aspects, the present simulation-based approaches may resemble those described in “Real-world data validation of the PurIST pancreatic ductal adenocarcinoma gene expression classifier and its prognostic implications,” Wenric, et al., medRxiv 2023.02.23.23286356; doi: https://doi.org/10.1101/2023.02.23.23286356, hereby incorporated by reference in its entirety for all purposes.

Samples with confidence scores below a threshold may be flagged as “no-calls” and not assigned a subtype; further, in some aspects, these no-call samples may be censored from analytical and clinical validations of the algorithm and may not have an associated subtype reported to clinicians or patients.

In one aspect, the present techniques quantify confidence by using reference samples (e.g., universal human reference RNA (UHR)). The present techniques may compare noise from many (e.g., thousands or more) sequencing runs to parameterize noise specific to the sequencers used during those sequencing runs. For example, a standard deviation of UHR 20 k genes may be used to set the coefficients (this is sequencer dependent). This enables the present techniques to develop a dynamic confidence threshold for each gene. Examples of sequencers include Illumina HiSeq 4000 and Illumina NovaSeq 6000. Methods of next-generation sequencing for use in accordance with methods described herein are disclosed in Shendure 2008 Nat. Biotechnology 26:1135-1145 and Fullwood et al. 2009 Genome Res. 19:521-532, which are each hereby incorporated by reference in their respective entireties, for all purposes. Next generation sequencing methods well known in the art include synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), nanopore sequencing (Oxford Nanopore Technologies), or paired-end sequencing. In some embodiments, massively parallel sequencing is performed using sequencing-by-synthesis with reversible dye terminators.

The present techniques are applicable to any RNA expression model that is used in a discretized fashion, and any sequencer that generates data (e.g., a set of quantified RNA expression values) that can be compared, enabling variance to be computed. Values whose variance may be quantified include gene expression levels, protein expression levels, enzyme activity, etc. However, the present techniques may be applicable to other continuous values.

In some aspects, the present techniques may be offered to another party, for example via a platform-as-a-service (PaaS) offering, software-as-a-service (Saas), a white labeled service, etc. Such other parties may include pharmaceutical companies, biotechnical companies, contract research organizations, startups (e.g., personalized medicine firms), healthcare analytics companies, software companies, pharmacogenomics companies, healthcare consultancies, non-profit research organizations, etc.

### Exemplary Computing Environments

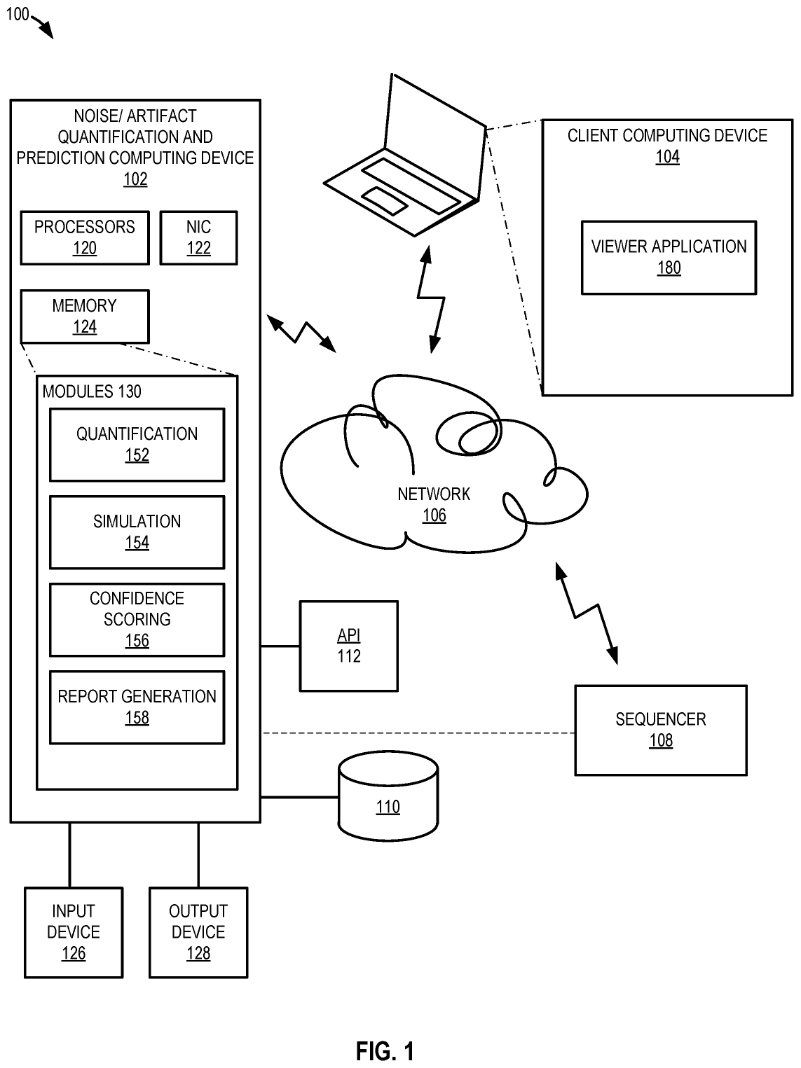

FIG. 1 illustrates an exemplary computing environment 100 for performing the present techniques, according to some aspects. The environment 100 may include computing resources for quantifying noise and other artifacts, and for predicting whether the noise/artifacts will result in classification indeterminacy, and for further processing/computations based on such predictions.

The computing environment 100 may include a noise/artifact quantification and prediction computing device 102, a client computing device 104, an electronic network 106, a sequencer system 108 and an electronic database 110. The prediction computing device 102 may include an application programming interface 112 that enables programmatic access to the prediction computing device 102. The components of the computing environment 100 may be communicatively connected to one another via the electronic network 106, in some aspects. Each will now be described in greater detail.

The noise/artifact quantification and prediction computing device 102 may implement, inter alia, receipt/storage/preprocessing of quantified data (e.g., from the sequencer 108), simulation-based algorithms, processing of quantified data, predictive tasks and related operations. In some aspects, the noise/artifact quantification and prediction computing device 102 may be implemented as one or more computing devices (e.g., one or more servers, one or more laptops, one or more mobile computing devices, one or more tablets, one or more wearable devices, one or more cloud-computing virtual instances, etc.). The noise/artifact quantification and prediction computing device 102 may include one or more processors 120, one or more network interface controllers 122, one or more memories 124, an input device 126 and an output device 128.

In some aspects, the one or more processors 120 may include one or more central processing units, one or more graphics processing units, one or more field-programmable gate arrays, one or more application-specific integrated circuits, one or more tensor processing units, one or more digital signal processors, one or more neural processing units, one or more RISC-V processors, one or more coprocessors, one or more specialized processors/accelerators for artificial intelligence or machine learning-specific applications, one or more microcontrollers, etc.

The quantitative measures prediction computing device 102 may include one or more network interface controllers 122, such as Ethernet network interface controllers, wireless network interface controllers, etc. The network interface controllers 122 may include advanced features, in some aspects, such as hardware acceleration, specialized networking protocols, etc.

The memories 124 of the quantitative measures prediction computing device 102 may include volatile and/or non-volatile storage media. For example, the memories 124 may include one or more random access memories, one or more read-only memories, one or more cache memories, one or more hard disk drives, one or more solid-state drives, one or more non-volatile memory express, one or more optical drives, one or more universal serial bus flash drives, one or more external hard drives, one or more network-attached storage devices, one or more cloud storage instances, one or more tape drives, etc.

The memories 124 may have stored thereon one or more modules 130, for example, as one or more sets of computer-executable instructions. In some aspects, the modules 130 may include additional storage, such as one or more operating systems (e.g., Microsoft Windows, GNU/Linux, Mac OSX, etc.). The operating systems may be configured to run the modules 130 during operation of the noise/artifact quantification and prediction computing device 102—for example, the modules 130 may include additional modules and/or services for receiving and processing quantitative data. The modules 130 may be implemented using any suitable computer programming language(s) (e.g., Python, JavaScript, C, C++, Rust, C#, Swift, Java, Go, LISP, Ruby, Fortran, etc.).

The modules 130 may include a quantification module 152, a simulation module 154, a confidence scoring module 156 and a report generation module 158. In some aspects, more or fewer modules 130 may be included. The modules 130 may be configured to communicate with one another (e.g., via inter-process communication, via a bus, message queue, sockets, etc.).

The quantification module 152 may collect data comprising continuous data values during an experimentation process, such as during the execution of one or more sequencers operated by the sequencer system 108. The quantification module 152 may include sets of computer-executable instructions for collecting and storing the continuous data values, for example, as a data stream. The quantification module 152 may store the collected continuous data values in the electronic database 110. The quantification module 152 may include instructions for pre-processing the continuous data values prior to storing it, and for associating the continuous data values with other data or metadata (e.g., sample run date/time, batch number, sample identifier, etc.).

### Exemplary Computer-Implemented Confidence Score Generation

The simulation module 154 may include computer-executable instructions for processing the collected continuous data values to generate confidence scores. For example, the simulation module 154 may include one or more algorithm for generating a predicted standard deviations for observed values. For example, the simulation module 154 may perform linear model computations.

In the case of the continuous data values corresponding to expression values for one or more features (e.g., one or more genes), the simulation module 154 may generate a predicted standard deviation by computing a linear model defined as

$$Predicted\_standard \_deviation ∼ a * sqrt ( log 10 ( observed\_expression ) ) - b ,$$

where constants a and b are determined by fitting the model to a dataset comprising a number (e.g., 1000) of replicate runs of a single sample (e.g., a universal human reference sample) on an RNA-Seq pipeline. For example, for one particular sequencing machine model, the constants a and b may be −0.08344563 and 0.2120142, respectively. The simulation module 154 may compute the standard deviation of each observed expression value for each algorithm feature (e.g., gene) using the linear model. The linear model may predict standard deviation based on mean expression alone and may be agnostic to gene identity.

In some aspects, the one or more features may be those of PurIST, a k-top scoring pairs (k-TSP) gene signature model that assesses rank-based expression levels of 16 genes sorted into 8 gene pairs from a single sample to estimate the likelihood of that sample being basal. PurIST is described in “Purity Independent Subtyping of Tumors (PurIST), A Clinically Robust, Single-Sample Classifier for Tumor Subtyping in Pancreatic Cancer”; Clinical Cancer Research: An Official Journal of the American Association for Cancer; Research 26 (1): 82-92.Rashid et al. 2020, hereby incorporated by reference in its entirety, for all purposes. It should be appreciated that other subtyping algorithms (e.g., k-TSP based algorithms or other algorithms, single-gene models, and subtyping models including those derived from hierarchical clustering, consensus clustering, NMF, k-means, kNN and any other subtyping model making use of gene expression data to classify and label samples) may benefit from the present techniques, in addition to, or alternatively from PurIST. Generally, any model that uses any number of continuous values subject to measurement error (e.g., gene expression values) to determine discrete subtypes may be aided by the present techniques.

Other classifiers that may be used include those discussed in U.S. Pat. No. 11,527,323 entitled “Systems and methods for multi-label cancer classification,” filed on May 12, 2020 and hereby incorporated by reference herein in its entirety; and those discussed in U.S. Patent Application Publication No. 20220154284, entitled “Determination of cytotoxic gene signature and associated systems and methods for response prediction and treatment,” filed on Nov. 19, 2021, and incorporated hereby in reference in its entirety. In some aspects, the classifiers discussed in U.S. patent application Ser. No. 17/738,935, entitled “Techniques for resolving discordant her2 status in pathology images for diagnosing breast cancer” and filed on May 6, 2022, hereby incorporated by reference in its entirety, may be used. The present techniques may be extended to any subtyping algorithm, and PurIST is merely one example. For example, in some aspects, the present techniques may be used to quantify variance in gene co-expression analysis, differential expression analysis, gene network inference techniques, etc.

Next, the simulation module 154 may simulate a number of classification values, by drawing a number (e.g., 16) of gene log-transformed expression values from a number (e.g., 16) of normal distributions defined using the observed log-transformed expression values as their means along with their respective predicted standard deviations. The simulation module 154 may then apply the classifier to the simulated classifications to determine a simulated predicted classification. This process may be performed numerous times, for example, from simulations 1 . . . . N, a new set of simulated expression values may be selected from the normal distribution.

The simulation module 154 may store the simulated expression values and the simulated predicted classification in the electronic database 110, in some aspects. The simulation module 154 may also transmit the simulated expression values and the simulated predicted classification to another element of the environment 100, or beyond, in some aspects. Next, the simulation module 154 may repeat the simulation procedure a number (e.g., 10,000 or more) of times, and the frequencies of predicted subtypes may be counted.

The confidence scoring module 156 may include computer-executable instructions for computing one or more confidence scores. For example, the module 156 may compute one or more confidence scores by determining a ratio of each given classification to a number of total simulations. For example, if the simulation is performed by the simulation module 154 for a given sample is 10,000 times, and 9000 of the classifications classify the sample in a bin X, then the score would be 0.90 for bin X. As noted, the classifier may be a binary classifier (in which the majority of classifications may constitute the score) or a multi classifier, in which a sample is classified into two or more bins.

Generally, for a binary classifier embodiment, a score of 0.50 is a very low confidence score, being equivalent to a coin flip, while a 1.0 score is a very high confidence score, with zero discordance expected. The confidence scoring module 156 may store the generated confidence score(s) for a given sample in the electronic database 110, in some aspects. The confidence scoring module 156 may provide the one or more confidence scores to one or more other elements of the environment 100, such as the report generation module 158.

### Exemplary Computer-Implemented Rules Engine

The present techniques may establish a confidence score cutoff, wherein any confidence score less than that score is considered a “no call,” meaning that the confidence level is below a predetermined threshold (e.g., 0.85). In some aspects, a no call confidence score may be used by the report generation module 158 to censor or abort communication to patients or others about corresponding classifications. The report generation module 158 may also allow “no call” classifications to be included in reports, but only with disclaimer language, “low confidence” flags, or other visual/graphical indicia, to advise the reader/viewer accordingly as to the potentially discordant status of the classification.

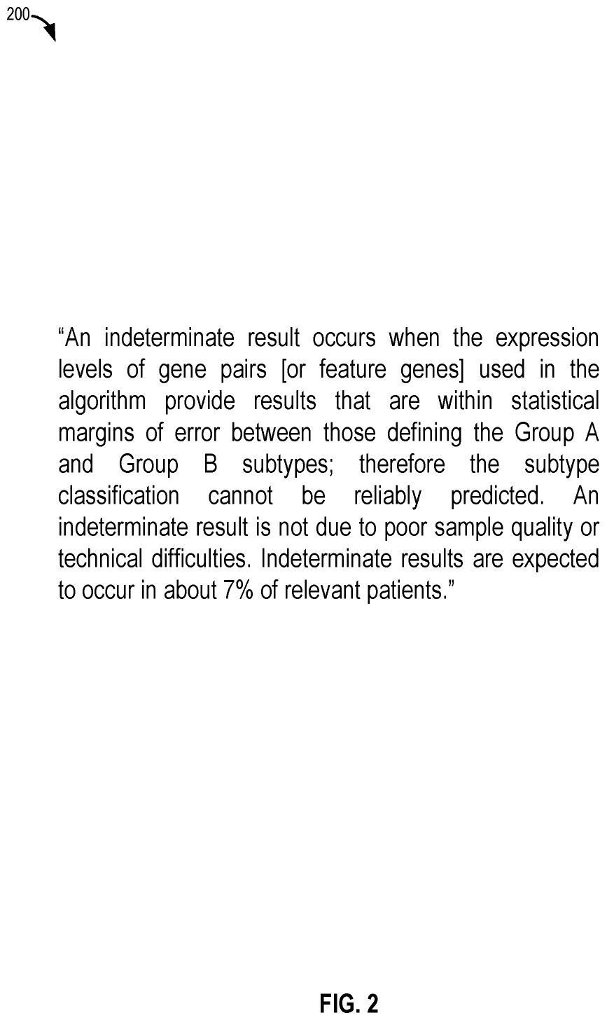

In some aspects, the report generation module 158 may generate static reports that include predictions regarding the confidence of classifications of patient data, as shown in FIG. 2. For example, these static reports may take the form of text documents, digital presentations, JSON documents, PDF documents, word processing documents, etc.

In some aspects, the report generation module 158 may include a rules engine for programmatically generating filtered sets of classification results based on confidence scores. For example, the electronic database 110 may include a vector or table of information corresponding to a sequencing run of a patient's tissue. In that case, the rows of the table may each include a respective gene name (e.g., as column 1) and an associated respective expression level value (e.g., column 2). The report generation module 158 may process that vector of information and perform its classification prediction routines as discussed above, storing the results in the electronic database 110.

Later, the prediction computing device 102 may receive, e.g., via the viewer application 180, an electronic request (e.g., an HTTP POST or GET request) for classification results. The 102 may inspect the request to determine the identity of the requestor. In some aspects, the prediction computing device 102 may include a role module (not depicted) that accepts the identity of the requestor (e.g., as a session identifier, a login identifier, an authentication credential, etc.) and determines, based on that identity identifier, a role of the user (e.g., patient, clinician, research, etc.).

The rules engine of the report generation module 158 may include one or more sets of computer-executable instructions specifying which predictions the role is entitled to. The report generation module 158 may generate a query specific to the role, and issue that query against the database 110, to retrieve only those results that the user is entitled to, based on the user's role. The report generation module 158 may then generate an electronic response (e.g., an HTTP response) to the electronic request, and transmit the filtered results to the user. In some aspects, the report generation module 158 may include “no call” classification results in this response, but only after additional rules in the rules engine annotate those results as “no call” results using a flag or other indicia, as discussed above. In some aspects, a user (e.g., a clinician) may set a preference via the viewer application 180 that affects the way that “no call” results are identified in data sets.

The present reporting techniques represent an advantageous improvement over conventional classification confidence techniques, especially because the environment 100 can be plugged into a larger computational pipeline, to provide a completely automated solution for annotation and sharing of clinical results with no-call annotations or filtering. For example, instead of the user-initiated request/response interaction discussed above, in some aspects, the application programming interface 112 may receive authenticated requests from one or more scheduled computer programs (e.g., a script that runs periodically on a remote computing device). Such script programs may be configured to have a suitable role, so that the information they receive in responses from the application programming interface 112 is appropriately filtered/annotated.

The report generation module 158 may include computer-executable instructions for generating machine-readable results via the application programming interface 112. For example, the application programming interface 112 may generate a structured output (e.g., results encoded in JavaScript Object Notation, XML, etc.), so that a calling client of the application programming interface 112 can easily extract data from the results, including data corresponding to the confidence score of the data.

The client computing device 104 may be accessed by a user to view results generated by the prediction computing device 102. For example, a user may access a mobile device, laptop device, thin client, etc. embodied as the client computing device 104 to view simulation and confidence scoring results, and/or reports, with respect to a sample whose continuous values were processed by the prediction computing device 102.

Information from the prediction computing device 102 may be transmitted via the network 106 (e.g., for display via the viewer application 180).

The electronic network 106 may communicatively couple the elements of the environment 100. The network 106 may include public network(s) such as the Internet, a private network such as a research institution or corporation private network, and/or any combination thereof. The network 106 may include a local area network (LAN), a wide area network (WAN), a cellular network, a satellite network, and/or other network infrastructure, whether wireless or wired.

In some aspects, the network 106 may be communicatively coupled to and/or part of a cloud-based platform (e.g., a cloud computing infrastructure). The network 106 may utilize communications protocols, including packet-based and/or datagram-based protocols such as Internet protocol, transmission control protocol, user datagram protocol, and/or other types of protocols. The network 106 may include one or more devices that facilitate network communications and/or form a hardware basis for the networks, such as one or more switches, one or more routers, one or more gateways, one or more access points (such as a wireless access point), one or more firewalls, one or more base stations, one or more repeaters, one or more backbone devices, etc.

The sequencer system 108 may include a next generation sequencer, such as a RNA sequencing whole exome capture transcriptome assay. As discussed in U.S. Pat. No. 11,043,283, entitled “Systems and methods for automating RNA expression calls in a cancer prediction pipeline,” filed Dec. 4, 2020 and in U.S. Pat. No. 11,367,508, entitled “Systems and methods for detecting cellular pathway dysregulation in cancer,” filed Aug. 14, 2020—both of which are hereby incorporated by reference in their respective entireties, for all purposes—in various embodiments, each transcriptome value set may be generated by processing a patient or tumor organoid sample through RNA whole exome next generation sequencing (NGS) to generate RNA sequencing data, and the RNA sequencing data may be processed by a bioinformatics pipeline to generate a RNA-seq expression profile for each sample. The patient sample may be a tissue sample or blood sample containing cancer cells. In more detail, RNA may be isolated from blood samples or tissue sections using commercially available reagents, for example, proteinase K, TURBO DNase-I, and/or RNA clean XP beads. The isolated RNA may be subjected to a quality control protocol to determine the concentration and/or quantity of the RNA molecules, including the use of a fluorescent dye and a fluorescence microplate reader, standard spectrofluorometer, or filter fluorometer. cDNA libraries may be prepared from the isolated RNA, purified, and selected for cDNA molecule size selection using commercially available reagents, for example Roche KAPA Hyper Beads. In another example, a New England Biolabs (NEB) kit may be used. cDNA library preparation may include the ligation of adapters onto the cDNA molecules. For example, UDI adapters, including Roche SeqCap dual end adapters, or UMI adapters (for example, full length or stubby Y adapters) may be ligated to the cDNA molecules. The sequence of nucleotides in the adapters may be specific to a sample in order to distinguish between sequencing data obtained for different samples. In this example, adapters are nucleic acid molecules that may serve as barcodes to identify cDNA molecules according to the sample from which they were derived and/or to facilitate the next generation sequencing reaction and/or the downstream bioinformatics processing. cDNA libraries may be amplified and purified using reagents, for example, Axygen MAG PCR clean up beads. Then the concentration and/or quantity of the cDNA molecules may be quantified using a fluorescent dye and a fluorescence microplate reader, standard spectrofluorometer, or filter fluorometer. cDNA libraries may be pooled and treated with reagents to reduce off-target capture, for example Human COT-1 and/or IDT xGen Universal Blockers, before being dried in a vacufuge. Pools may then be resuspended in a hybridization mix, for example, IDT xGen Lockdown, and probes may be added to each pool, for example, IDT xGen Exome Research Panel v1.0 probes, IDT xGen Exome Research Panel v2.0 probes, other IDT probe panels, Roche probe panels, or other probes. Pools may be incubated in an incubator, PCR machine, water bath, or other temperature modulating device to allow probes to hybridize. Pools may then be processed with Streptavidin-coated beads, or another means for capturing hybridized cDNA-probe molecules, especially cDNA molecules representing exons of the human genome. In some embodiments, polyA capture may be used. Pools may be amplified and purified once more using commercially available reagents, for example, the KAPA HiFi Library Amplification kit and Axygen MAG PCR clean up beads, respectively. The cDNA library may be analyzed to determine the concentration or quantity of cDNA molecules, for example by using a fluorescent dye (for example, PicoGreen pool quantification) and a fluorescence microplate reader, standard spectrofluorometer, or filter fluorometer. The cDNA library may also be analyzed to determine the fragment size of cDNA molecules, which may be done through gel electrophoresis techniques and may include the use of a device such as a LabChip GX Touch. Pools may be cluster amplified using a kit (for example, Illumina Paired-end Cluster Kits with PhiX-spike in). In one example, the cDNA library preparation and/or whole exome capture steps may be performed with an automated system, using a liquid handling robot (for example, a SciClone NGSx). The amplification may be performed on a device, for example, an Illumina C-Bot2, and the resulting flow cell containing amplified target-captured cDNA libraries may be sequenced on a next generation sequencer, for example, an Illumina HiSeq 4000 or an Illumina NovaSeq 6000 to a unique on-target depth selected by the user, for example, 300x, 400x, 500x, 10,000x, etc. The next generation sequencer may generate a FASTQ file for each patient sample. Each FASTQ file contains reads that may be paired-end or single reads, and may be short-reads or long-reads, where each read shows one detected sequence of nucleotides in an mRNA molecule that was isolated from the patient sample, inferred by using the sequencer to detect the sequence of nucleotides contained in a cDNA molecule generated from the isolated mRNA molecules during library preparation. Each read in the FASTQ file is also associated with a quality rating. The quality rating may reflect the likelihood that an error occurred during the sequencing procedure that affected the associated read. The adapters may facilitate the binding of the cDNA molecules to anchor oligonucleotide molecules on the sequencer flow cell and may serve as a seed for the sequencing process by providing a starting point for the sequencing reaction. If two or more patient samples are processed simultaneously on the same sequencer flow cell, reads from multiple patient samples may be contained in the same FASTQ file initially and then divided into a separate FASTQ file for each patient. A difference in the sequence of the adapters used for each patient sample could serve the purpose of a barcode to facilitate associating each read with the correct patient sample and placing it in the correct FASTQ file. Each FASTQ file may be processed by a bioinformatics pipeline. In various embodiments, the bioinformatics pipeline may filter FASTQ data. Filtering FASTQ data may include correcting sequencer errors and removing (trimming) low quality sequences or bases, adapter sequences, contaminations, chimeric reads, overrepresented sequences, biases caused by library preparation, amplification, or capture, and other errors. Entire reads, individual nucleotides, or multiple nucleotides that are likely to have errors may be discarded based on the quality rating associated with the read in the FASTQ file, the known error rate of the sequencer, and/or a comparison between each nucleotide in the read and one or more nucleotides in other reads that has been aligned to the same location in the reference genome. Filtering may be done in part or in its entirety by various software tools. FASTQ files may be analyzed for rapid assessment of quality control and reads, for example, by a sequencing data QC software such as AfterQC, Kraken, RNA-SeQC, FastQC, (see Illumina, BaseSpace Labs or https://world wide web address illumina.com/products/by-type/informatics-products/basespace-sequence-hub/apps/fastqc.html), or another similar software program. For paired-end reads, reads may be merged. For each FASTQ file, each read in the file may be aligned to the location in the reference genome having a sequence that best matches the sequence of nucleotides in the read. There are many software programs designed to align reads, for example, Bowtie, Burrows Wheeler Aligner (BWA), programs that use a Smith-Waterman algorithm, etc. Alignment may be directed using a reference genome (for example, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. The alignment may take RNA splice sites into account. The alignment may generate a SAM file, which stores the locations of the start and end of each read in the reference genome and the coverage (number of reads) for each nucleotide in the reference genome. The SAM files may be converted to BAM files, BAM files may be sorted, and duplicate reads may be marked for deletion. In one example, kallisto software may be used for alignment and RNA read quantification (see Nicolas L Bray, Harold Pimentel, Pall Melsted and Lior Pachter, Near-optimal probabilistic RNA-seq quantification, Nature Biotechnology 34, 525-527 (2016), doi: 10.1038/nbt.3519). In an alternative embodiment, RNA read quantification may be conducted using another software, for example, Sailfish or Salmon (see Rob Patro, Stephen M. Mount, and Carl Kingsford (2014) Sailfish enables alignment-free isoform quantification from RNA-seq reads using lightweight algorithms. Nature Biotechnology (doi: 10.1038/nbt.2862) or Patro, R., Duggal, G., Love, M. I., Irizarry, R. A., & Kingsford, C. (2017). Salmon provides fast and bias-aware quantification of transcript expression. Nature Methods, incorporated by reference herein in its entirety for all purposes). These RNA-seq quantification methods may not require alignment. There are many software packages that may be used for normalization, quantitative analysis, and differential expression analysis of RNA-seq data. For each gene, the raw RNA read count for a given gene may be calculated. The raw read counts may be saved in a tabular file for each sample, where columns represent genes and each entry represents the raw RNA read count for that gene. In one example, kallisto alignment software calculates raw RNA read counts as a sum of the probability, for each read that the read aligns to the gene. Raw counts are therefore not integers in this example. Raw RNA read counts may then be normalized to correct for GC content and gene length, for example, using full quantile normalization and adjusted for sequencing depth, for example, using the size factor method. In one example, RNA read count normalization is conducted according to the methods disclosed in U.S. patent application Ser. No. 16/581,706 or PCT19/52801, titled Methods of Normalizing and Correcting RNA Expression Data and filed Sep. 24, 2019, incorporated by reference herein in their respective entireties. The rationale for normalization is the number of copies of each cDNA molecule in the sequencer may not reflect the distribution of mRNA molecules in the patient sample. For example, during library preparation, amplification, and capture steps, certain portions of mRNA molecules may be over or under-represented due to artifacts that arise during various aspects of priming of reverse transcription caused by random hexamers, amplification (PCR enrichment), rRNA depletion, and probe binding and errors produced during sequencing that may be due to the GC content, read length, gene length, and other characteristics of sequences in each nucleic acid molecule. Each raw RNA read count for each gene may be adjusted to eliminate or reduce over- or under-representation caused by any biases or artifacts of NGS sequencing protocols. Normalized RNA read counts may be saved in a tabular file for each sample, where columns represent genes and each entry represents the normalized RNA read count for that gene. The electronic database 110 may include one or more suitable electronic databases for storing and retrieving data, such as relational database (e.g., MySQL databases, Oracle databases, Microsoft SQL Server databases, PostgreSQL databases, etc.). The electronic database 110 may be a NoSQL database, such as a key-value store, a graph database, a document store, etc. The electronic database 110 may be an object-oriented database, a hierarchical database, a spatial database, a time-series database, an in-memory database, etc. In some aspects, some or all of the electronic database 110 may be distributed.

In operation, one or more sequencer runs may be performed using the sequencer 108, either by the company operating/controlling the environment 100 or by another party. The results of the sequencer are received by the prediction computing device 102, which performs quantification 152 of the results, and optionally, storage of the quantified results. Next, the simulation module 154 processes the results, to generate confidence scores for the results. The confidence scores may be stored in association with the stored quantified results in the database 110, for example. Next the prediction computing device 102 receives a request for data. The prediction computing device 102 provides the data, after filtering and/or annotating the data by applying rules to parameters included in the request for data. Overall, the process results in a quality control check that prevents clinicians from misinterpreting low confidence data, and prevents patients from losing confidence in the sequencing process due to the potential for passing along indeterminate results-which is advantageously largely eliminated using the present techniques, because the quality control check is performed before data is ever provided to patients.

### Exemplary Computer-Implemented Report Generation

FIG. 2 depicts exemplary no-call language 200 that may be generated by the rules engine of the report generation module 158, in some aspects. For example, the no-call language of FIG. 2 may be the language generated when the rules engine determines, via the application programming interface 112 that the requestor is a patient computing device, or that the user making the request is logged in and has a role of patient.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### Exemplary Computer-Implemented Method Aspects

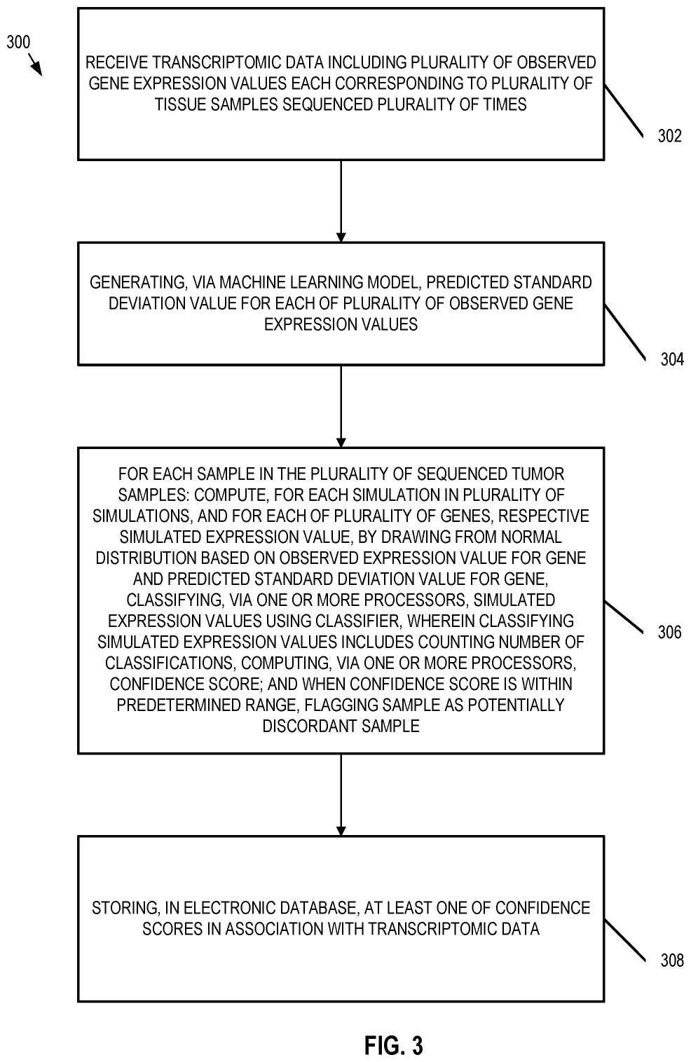

FIG. 3 depicts an exemplary computer-implemented quality control method 300 for predicting whether a continuous value classifier will generate discordant classifications of a sample, according to some aspects. The method may be performed by one or more of the components of environment 100 of FIG. 1, in some aspects.

The method 300 may include receiving, via one or more processors, transcriptomic data including a plurality of observed gene expression values each corresponding to one of a plurality of samples sequenced a plurality of times (block 302). The samples may have originated from various sources, including but not limited to, tissue samples, commercially available reference RNA, manufactured RNA molecules, etc. The method 300 may include generating, via a machine learning model, a predicted standard deviation value for each of the plurality of observed gene expression values (block 304). For example, the machine learning model may be a Bayesian model, a supervised machine learning model or an unsupervised machine learning model.

The method 300 may include for each sample in the plurality of samples (e.g., sequenced tumor samples): (i) computing, for each simulation in a plurality of simulations, and for each of a plurality of genes, a respective simulated expression value, by drawing from a normal distribution based on an observed expression value for the gene and the predicted standard deviation value for the gene; (ii) classifying, via one or more processors, the simulated expression values using the classifier, wherein classifying the simulated expression values includes counting a number of classifications; (iii) computing, via one or more processors, a confidence score; and (iv) when the confidence score is within a predetermined range, flagging the sample as a potentially discordant sample (block 306).

The method 300 may include storing, in an electronic database, at least one of the confidence scores in association with the transcriptomic data (block 308).

In some aspects, the method 300 may include generating, via one or more processors, a static digital report including at least one classification result. In some aspects, generating the static digital report including the at least the one classification result may include censoring a no-call classification result.

In some aspects, the method 300 may include receiving, via an application programming interface, a request for sequence data (e.g., RNA sequence data); processing, via one or more processors, the request using a rules engine to determine an identity of the sender of the request; determining, via one or more processors, a role of the identity of the sender of the request; configuring, via one or more processors, a rules engine based on the role of the identity of the sender of the request; generating, via one or more processors, modified sequence data by processing the sequence data using the configured rules engine; and/or transmitting, via electronic network, the modified sequence data to a computing device of the sender of the request. In some aspects, generating the modified sequence data by processing the sequence data using the configured rules engine may generate censored results when the role of the identity of the sender of the request is a patient. In some aspects, censored results may be generated using the method 300 in other circumstances (e.g., when the results will be shared with another party that is not authorized to view uncensored results, for example due to data privacy laws). The rules engine may include additional rules governing these scenarios, in some aspects.

In some aspects, generating the modified sequence data by processing the sequence data using the configured rules engine may include generating annotated results when the role of the identity of the sender of the request is a clinician. Specifically, the annotated results may include a binary indication (e.g., call/no call) and/or additional indicia of the quality control check on the results. For example, the additional indicia may include or relate to the predicted standard deviation values, the classifications of the simulated expression values (e.g., count information), and/or the confidence scores. Whereas this information may be censored or not displayed for a patient, the physician or another viewer may have more insight into the underlying quality control techniques, and the information may prove useful for interpretation. In some aspects, the annotations may include an indication of a patient therapy, including one or more alternative therapy as discussed below.

In some aspects, the method 300 may be performed automatically as part of a bioinformatics pipeline, wherein the role of the sender of the request is a programmatic script; and wherein generating the modified sequence data by processing the sequence data may include programmatically generating a structured output corresponding to the modified sequence data, wherein the structured output includes the modified sequence data and one or more confidence scores corresponding to the modified sequence data. For example the structured output may be an XML output, a JSON output, a CSV output or another structured data format. The method 300 may include serving the structured output via an authenticated API (e.g., the API 112) to a requesting client (e.g., the client computing device 104) via an electronic network (e.g., the network 106) in some aspects.

In some aspects, the number of classifications is two or more. For example, the method 300 may include classifications with respect to two subtypes, a subtype A and a subtype B, as discussed herein. In some aspects, the classifications may be multi-stage classifications.

In some aspects, the method 300 may include determining, based on flagging the sample as the potentially discordant sample, an alternative therapy that may be an appropriate match for the patient corresponding to the sample. The alternative therapy may include at least one of: (i) a first-line therapy or (ii) a second-line therapy. The alternative therapy may include at least one of: (i) a chemotherapeutic drug therapy, (ii) a radiation therapy, (iii) a surgery therapy, (iv) a therapy in a different class, (v) a molecular inhibitor therapy, (vi) an antibody therapy, (vii) a recombinant nucleic acid therapy, (viii) an engineered immune cell therapy, (ix) a checkpoint inhibitor therapy, (x) a cytokine therapy, (xi) a cancer treatment vaccine therapy, (xii) a CAR-cell therapy; or (xiii) an oncolytic virus therapy.

Additional exemplary therapeutic agents that can be used with the methods described herein are provided in Table 2. Additional alternative therapies are discussed in U.S. Pat. No. 11,415,571, entitled “Large scale organoid analysis,” filed on Dec. 7, 2020, and incorporated herein by reference in its entirety for all purposes.

TABLE 2

| Agent | Target | Pathway | Formula |
|---|---|---|---|
| Veliparib (ABT-888) | PARP | DNA Damage | C₁₃H₁₆N₄O |
| Selumetinib (AZD6244) | MEK | MAPK | C₁₇H₁₅BrClFN₄O3 |
| PD184352 (CI-1040) | MEK | MAPK | C₁₇H₁₄ClF₂IN₂O₂ |
| PD0325901 | MEK | MAPK | C₁₆H₁₄F₃IN₂O₄ |
| Tozasertib (VX-680, | Aurora Kinase | Cell Cycle | C₂₃H₂₈N₈OS |
| MK-0457) | | | |
| Y-27632 2HCl | Autophagy, ROCK | Cell Cycle | C₁₄H₂₃Cₗ₂N₃O |
| Olaparib (AZD2281, | PARP | DNA Damage | C₂₄H₂₃FN₄O₃ |
| Ku-0059436) | | | |
| SL-327 | MEK | MAPK | C₁₆H₁₂F₃N₃S |
| SB431542 | TGF-beta/Smad | TGF-beta/Smad | C₂₂H₁₆N₄O₃ |
| MK-2206 2HCl | Akt | PI3K/Akt/mTOR | C₂₅H₂₃Cₗ₂N₅O |
| Refametinib (RDEA119, | MEK | MAPK | C₁₉H₂₀F₃IN₂O₅S |
| Bay | | | |
| 86-9766) | | | |
| KU-55933 (ATM Kinase | ATM/ATR | DNA Damage | C₂₁H₁₇NO₃S₂ |
| Inhibitor) | | | |
| GSK1904529A | IGF-1R | Protein Tyrosine | C₄₄H₄₇F₂N₉O₅S |
| | | Kinase | |
| PF-04217903 | c-Met | Protein Tyrosine | C₁₉H₁₆N₈O |
| | | Kinase | |
| U0126-EtOH | MEK | MAPK | C₂₀H₂₂N₆OS₂ |
| BI 2536 | PLK | Cell Cycle | C28H39N7O3 |
| JNJ-38877605 | c-Met | Protein Tyrosine | C19H13F2N7 |
| | | Kinase | |
| Odanacatib (MK-0822) | Cysteine Protease | Proteases | C₂₅H₂₇F₄N₃O₃S |
| Alisertib (MLN8237) | Aurora Kinase | Cell Cycle | C27H20ClFN4O4 |
| Barasertib (AZD1152- | Aurora Kinase | Cell Cycle | C26H30FN7O3 |
| HQPA) | | | |
| CP-724714 | EGFR, HER2 | Protein Tyrosine | C27H27N5O3 |
| | | Kinase | |
| TGX-221 | PI3K | PI3K/Akt/mTOR | C21H24N4O2 |
| WZ4002 | EGFR | Protein Tyrosine | C25H27ClN6O3 |
| | | Kinase | |
| BIBR 1532 | Telomerase | DNA Damage | C21H17NO3 |
| Anastrozole | Aromatase | Endocrinology & | C17H19N5 |
| | | Hormones | |
| Aprepitant | Substance P | Others | C23H21F7N4O3 |
| TAK-700 (Orteronel) | P450 (e.g. CYP17) | Metabolism | C18H17N3O2 |
| PFI-1 (PF-6405761) | Epigenetic Reader | Epigenetics | C16H17N3O4S |
| | Domain | | |
| KU-0063794 | mTOR | PI3K/Akt/mTOR | C25H31N5O4 |
| CHIR-99021 (CT99021) | GSK-3 | PI3K/Akt/mTOR | C22H18Cl2N8 |
| WYE-354 | mTOR | PI3K/Akt/mTOR | C24H29N7O5 |
| TG100-115 | PI3K | PI3K/Akt/mTOR | C18H14N6O2 |
| Aurora A Inhibitor I | Aurora Kinase | Cell Cycle | C31H31ClFN7O2 |
| Ispinesib (SB-715992) | Kinesin | Cytoskeletal | C30H33ClN4O2 |
| | | Signaling | |
| Zibotentan (ZD4054) | Endothelin Receptor | GPCR & G | C19H16N6O4S |
| | | Protein | |
| Safinamide Mesylate | MAO | Metabolism | C18H23FN2O5S |
| GSK429286A | ROCK | Cell Cycle | C21H16F4N4O2 |
| Pimasertib (AS-703026) | MEK | MAPK | C15H15FIN3O3 |
| Tadalafil | PDE | Metabolism | C22H19N3O4 |
| Adavosertib (MK-1775) | Wee1 | Cell Cycle | C27H32N8O2 |
| CP-673451 | PDGFR | Protein Tyrosine | C24H27N5O2 |
| | | Kinase | |
| Selisistat (EX 527) | Sirtuin | Epigenetics | C13H13ClN2O |
| Dapagliflozin | SGLT | GPCR & G | C21H25ClO6 |
| | | Protein | |
| Nebivolol HCl | Adrenergic Receptor | Neuronal | C22H26ClF2NO4 |
| | | Signaling | |
| Pimobendan | PDE | Metabolism | C19H18N4O2 |
| AZD8055 | mTOR | PI3K/Akt/mTOR | C25H31N5O4 |
| KU-60019 | ATM/ATR | DNA Damage | C30H33N3O5S |
| Tie2 kinase inhibitor | Tie-2 | ProteinTyrosine | C26H21N3O2S |
| | | Kinase | |
| Apixaban | Factor Xa | Metabolism | C25H25N5O4 |
| Raltegravir (MK-0518) | Integrase | Microbiology | C20H21FN6O5 |
| PCI-34051 | HDAC | Epigenetics | C17H16N2O3 |
| Ambrisentan | Endothelin Receptor | GPCR & G | C22H22N2O4 |
| | | Protein | |
| SB743921 HCl | Kinesin | Cytoskeletal | C31H34Cl2N2O3 |
| | | Signaling | |
| AST-1306 | EGFR | Protein Tyrosine | C₃₁H₂₆ClFN₄O₅S |
| | | Kinase | |
| Sapitinib (AZD8931) | EGFR, HER2 | Protein Tyrosine | C23H25ClFN5O3 |
| | | Kinase | |
| GSK461364 | PLK | Cell Cycle | C27H28F3N5O2S |
| Mubritinib (TAK 165) | HER2 | Protein Tyrosine | C25H23F3N4O2 |
| | | Kinase | |
| UK 383367 | Procollagen C | Metabolism | C15H24N4O4 |
| | Proteinase | | |
| Cryptotanshinone | STAT | JAK/STAT | C19H20O3 |
| Icariin | PDE | Metabolism | C33H40O15 |
| OSI-027 | mTOR | PI3K/Akt/mTOR | C21H22N6O3 |
| Rabusertib | Chk | Cell Cycle | C18H22BrN5O3 |
| (LY2603618) | | | |
| URB597 | FAAH | Metabolism | C20H22N2O3 |
| A66 | PI3K | PI3K/Akt/mTOR | C17H23N5O2S2 |
| ICG-001 | Wnt/beta-catenin | Stem Cells & | C33H32N4O4 |
| | | Wnt | |
| PF-3845 | FAAH | Metabolism | C24H23F3N4O2 |
| Trametinib | MEK | MAPK | C26H23FIN5O4 |
| (GSK1120212) | | | |
| Ibrutinib (PCI-32765) | BTK | Angiogenesis | C25H24N6O2 |
| CHIR-124 | Chk | Cell Cycle | C23H22ClN5O |
| Mardepodect (PF- | PDE | Metabolism | C25H20N4O |
| 2545920) | | | |
| WAY-600 | mTOR | PI3K/Akt/mTOR | C28H30N8O |
| Nepicastat (SYN-117) | Hydroxylase | Metabolism | C14H16ClF2N3S |
| HCl | | | |
| RS-127445 | 5-HT Receptor | Neuronal | C17H16FN3 |
| | | Signaling | |
| CP-91149 | Phosphorylase | Metabolism | C21H22ClN3O3 |
| SB415286 | GSK-3 | PI3K/Akt/mTOR | C16H10ClN3O5 |
| GSK1070916 | Aurora Kinase | Cell Cycle | C30H33N7O |
| Niraparib (MK-4827) | PARP | DNA Damage | C19H20N4O |
| CHIR-98014 | GSK-3 | PI3K/Akt/mTOR | C20H17Cl2N9O2 |
| AMG-458 | c-Met | Protein Tyrosine | C30H29N5O5 |
| | | Kinase | |
| Tivantinib (ARQ 197) | c-Met | Protein Tyrosine | C23H19N3O2 |
| | | Kinase | |
| Canagliflozin | SGLT | GPCR & G | C24H25FO5S |
| | | Protein | |
| NVP-BVU972 | c-Met | Protein Tyrosine | C20H16N6 |
| | | Kinase | |
| MK-5108 (VX-689) | Aurora Kinase | Cell Cycle | C₂₂H₂₁ClFN₃O₃S |
| SB705498 | TRPV | Others | C17H16BrF3N4O |
| Vistusertib (AZD2014) | mTOR | PI3K/Akt/mTOR | C25H30N6O3 |
| A-803467 | Sodium Channel | Transmembrane | C19H16ClNO4 |
| | | Transporters | |
| Sirtinol | Sirtuin | Epigenetics | C26H22N2O2 |
| Ipatasertib (GDC-0068) | Akt | PI3K/Akt/mTOR | C24H32ClN5O2 |
| Sapanisertib (INK 128, | mTOR | PI3K/Akt/mTOR | C15H15N7O |
| MLN0128) | | | |
| Tyrphostin AG 879 | HER2 | Protein Tyrosine | C18H24N2OS |
| | | Kinase | |
| JNJ-1661010 | FAAH | Metabolism | C19H19N5OS |
| CTEP (RO4956371) | GluR | Neuronal | C19H13ClF3N3O |
| | | Signaling | |
| Alogliptin (SYK-322) | DPP-4 | Proteases | C18H21N5O2 |
| benzoate | | | |
| T0070907 | PPAR | DNA Damage | C12H8ClN3O3 |
| GW441756 | Trk receptor | Protein Tyrosine | C17H13N3O |
| | | Kinase | |
| SB742457 | 5-HT Receptor | Neuronal | C19H19N3O2S |
| | | Signaling | |
| ZM 323881 HCl | VEGFR | Protein Tyrosine | C22H19ClFN3O2 |
| | | Kinase | |
| GNF-2 | Bcr-Abl | Angiogenesis | C18H13F3N4O2 |
| Lumiracoxib | COX | Neuronal | C15H13ClFNO2 |
| | | Signaling | |
| JNJ-7777120 | Histamine Receptor | Neuronal | C14H16ClN3O |
| | | Signaling | |
| IOX2 | HIF | Angiogenesis | C19H16N2O5 |
| PF-4981517 | P450 (e.g. CYP17) | Metabolism | C26H32N8 |
| CHIR-99021 (CT99021) | GSK-3 | PI3K/Akt/mTOR | C22H19Cl3N8 |
| HCl | | | |
| Rivaroxaban | Factor Xa | Metabolism | C19H18ClN3O5S |
| Linagliptin | DPP-4 | Proteases | C25H28N8O2 |
| Azilsartan Medoxomil | RAAS | Endocrinology & | C30H24N4O8 |
| | | Hormones | |
| Sulfaphenazole | P450 (e.g. CYP17) | Metabolism | C15H14N4O2S |
| Sitagliptin phosphate | DPP-4 | Proteases | C16H20F6N5O6P |
| monohydrate | | | |
| Avanafil | PDE | Metabolism | C23H26ClN7O3 |
| Eprosartan Mesylate | RAAS | Endocrinology & | C24H28N2O7S2 |
| | | Hormones | |
| Carprofen | COX | Neuronal | C15H12ClNO2 |
| | | Signaling | |
| Saxagliptin hydrate | DPP-4 | Proteases | C18H27N3O3 |
| Daminozide | Histone Demethylase | Epigenetics | C6H12N2O3 |
| Bedaquiline fumarate | Anti-infection | Microbiology | C36H35BrN2O6 |
| JZL184 | Lipase | Metabolism | C27H24N2O9 |
| SC-514 | IκB/IKK | NF-κB | C9H8N2OS2 |
| (R)-Nepicastat HCl | Hydroxylase | Metabolism | C14H16ClF2N3S |
| Asunaprevir | HCV Protease | Proteases | C35H46ClN5O9S |
| Trelagliptin succinate | DPP-4 | Proteases | C22H26FN5O6 |
| Dabrafenib Mesylate | Raf | MAPK | C₂₄H₂₄F₃N₅O₅S₃ |
| Argatroban | Thrombin | Others | C23H38N6O6S |
| Monohydrate | | | |
| Sitagliptin | DPP-4 | Proteases | C16H15F6N5O |
| Raltegravir potassium | Integrase, HIV | Microbiology | C20H20FKN6O5 |
| | Protease | | |
| Alogliptin | DPP-4 | Proteases | C18H21N5O2 |
| Dasabuvir(ABT-333) | HCV Protease | Proteases | C26H27N3O5S |
| Ertugliflozin | SGLT2 | Ion-Channel | C22H25ClO7 |
| Rolapitant | NK1-receptor | GPCR | C25H26F6N2O2 |
| Dapagliflozin | SGLT | GPCR & G | C24H35ClO9 |
| propanediol | | | |
| monohydrate | | Protein | |
| Bedaquiline | tuberculosis | Immunology | C32H31BrN2O2 |
| Fruquintinib | VEGFRs | VEGFR | C21H19N3O5 |
| JNJ0966 | Others | Others | C16H16N4O2S2 |
| acalisib (GS-9820) | PI3K | PI3K/Akt/mTOR | C21H16FN7O |
| BRL-50481 | PDE | Metabolism | C9H12N2O4S |
| Canagliflozin | SGLT | GPCR & G | C48H52F2O11S2 |
| hemihydrate | | | |
| | | Protein | |
| JANEX-1 | JAK | JAK/STAT | C16H15N3O3 |
| Anagliptin | DPP-4 | Proteases | C19H25N7O2 |
| GSK 5959 | Epigenetic Reader Do | Epigenetics | C22H26N4O3 |
| Pitolisant | Histamine Receptor | Neuronal | C17H27Cl2NO |
| hydrochloride | | | |
| | | Signaling | |
| K 858 | Kinesin | Cytoskeletal | C13H15N3O2S |
| | | Signaling | |
| BAY-61-3606 | Syk | Angiogenesis | C20H20Cl2N603 |
| Stattic | STAT | JAK/STAT | C8H5NO4S |
| GSK2656157 | PERK | Apoptosis | C23H21FN6O |
| XL388 | mTOR | PI3K/Akt/mTOR | C23H22FN3O4S |
| LY2090314 | GSK-3 | PI3K/Akt/mTOR | C28H25FN6O3 |
| MK-8745 | Aurora Kinase | Cell Cycle | C20H19ClFN5OS |
| Tepotinib (EMD | c-Met | Protein Tyrosine | C29H28N6O2 |
| 1214063) | | | |
| | | Kinase | |
| SGC 0946 | Histone | Epigenetics | C28H40BrN7O4 |
| | Methyltransferase | | |
| GSK2334470 | PDK | PI3K/Akt/mTOR | C25H34N8O |
| IPA-3 | PAR | Cytoskeletal | C20H14O2S2 |
| | | Signaling | |
| VE-822 | ATM/ATR | PI3K/Akt/mTOR | C24H25N5O3S |
| (+)-JQ1 | Epigenetic Reader | Epigenetics | C23H25ClN4O2S |
| | Domain | | |
| PYR-41 | E1 Activating | Ubiquitin | C17H13N3O7 |
| TCID | DUB | Ubiquitin | C9H2Cl4O2 |
| DMH1 | TGF-beta/Smad | TGF-beta/Smad | C24H20N4O |
| ML347 | TGF-beta/Smad, ALK | TGF-beta/Smad | C22H16N4O |
| UNC1999 | Histone | Epigenetics | C33H43N7O2 |
| | Methyltransferase | | |
| SSR128129E | FGFR | Angiogenesis | C18H15N2NaO4 |
| Spebrutinib (CC-292, | BTK | Angiogenesis | C22H22FN5O3 |
| AVL-292) | | | |
| SKI II | S1P Receptor | GPCR & G | C15H11ClN2OS |
| | | Protein | |
| PF-543 | S1P Receptor | GPCR & G | C27H31NO4S |
| | | Protein | |
| CID755673 | Serine/threonin | Apoptosis | C12H11NO3 |
| | kinase, CaMK | | |
| 1-Azakenpaullone | GSK-3 | PI3K/Akt/mTOR | C15H10BrN3O |
| CNX-2006 | EGFR | Protein Tyrosine | C26H27F4N7O2 |
| | | Kinase | |
| Bisindolylmaleimide I | PKC | TGF-beta/Smad | C25H24N4O2 |
| (GF109203X) | | | |
| Thiamet G | Others | Others | C9H16N2O4S |
| Alvelestat (AZD9668) | Serine Protease | Proteases | C24H20F3N5O4S |
| RGFP966 | HDAC | Epigenetics | C21H19FN4O |
| UNC0642 | Histone | Epigenetics | C29H44F2N6O2 |
| | Methyltransferase | | |
| NVP-TNKS656 | PARP | DNA Damage | C27H34N4O5 |
| AGI-6780 | Dehydrogenase | Metabolism | C₂₁H₁₈F₃N₃O₃S₂ |
| Ro3280 | PLK | Cell Cycle | C27H35F2N7O3 |
| NMS-P937 | PLK | Cell Cycle | C24H27F3N8O3 |
| (NMS1286937) | | | |
| CNX-774 | BTK | Angiogenesis | C26H22FN7O3 |
| AZD1981 | GPR | Endocrinology & | C19H17ClN2O3S |
| | | Hormones | |
| SRPIN340 | Others | Others | C18H18F3N3O |
| 4μ8C | Others | Others | C11H8O4 |
| NMS-E973 | HSP (e g. HSP90) | Cytoskeletal | C22H22N4O7 |
| | | Signaling | |
| PFI-2 HCl | Histone | Epigenetics | C23H25F4N3O3S |
| | Methyltransferase | | |
| GSK2606414 | PERK | Apoptosis | C24H20F3N5O |
| IPI-3063 | PI3K | PI3K/Akt/mTOR | C25H25N7O2 |
| Atglistatin | Lipase | Metabolism | C17H21N3O |
| CGP 57380 | MNK | MAPK | C11H9FN6 |
| SB-3CT | MMP | Proteases | C15H14O3S2 |
| AR-A014418 | GSK-3 | PI3K/Akt/mTOR | C12H12N4O4S |
| NH125 | CaMK | Neuronal | C27H45IN2 |
| | | Signaling | |
| XEN445 | Lipase | Metabolism | C18H17F3N2O3R |
| LDC000067 | CDK | Cell Cycle | C18H18N4O3S |
| PI-1840 | Proteasome | Proteases | C22H26N4O3 |
| FTI 277 HCl | Transferase | Metabolism | C₂₂H₃₀CₗN₃O₃S₂ |
| Nexturastat A | HDAC | DNA Damage | C19H23N3O3 |
| ESI-09 | cAMP | GPCR & G | C16H15ClN4O2 |
| | | Protein | |
| HJC0350 | cAMP | GPCR & G | C15H19NO2S |
| | | Protein | |
| HO-3867 | STAT | JAK/STAT | C28H30F2N2O2 |
| JNK Inhibitor IX | JNK | MAPK | C20H16N2OS |
| Trelagliptin | DPP-4 | Proteases | C18H20FN5O2 |
| XMD8-92 | ERK | MAPK | C26H30N6O3 |
| A-366 | Histone | Epigenetics | C19H27N3O2 |
| | Methyltransferase | | |
| GSK-LSD1 2HCl | Histone Demethylase | Epigenetics | C14H22Cl2N2 |
| LLY-507 | Histone | Epigenetics | C36H42N6O |
| | Methyltransferase | | |
| Santacruzamate A | HDAC | DNA Damage | C15H22N2O3 |
| (CAY10683) | | | |
| CAY10603 | HDAC | DNA Damage | C22H30N4O6 |
| GSK1324726A (I- | Epigenetic Reader | Epigenetics | C25H23ClN2O3 |
| BET726) | | | |
| | Domain | | |
| SD-208 | TGF-beta/Smad | TGF-beta/Smad | C17H10ClFN6 |
| TH588 | MTH1 | DNA Damage | C13H12Cl2N4 |
| SB225002 | CXCR | GPCR & G | C13H10BrN3O4 |
| | | Protein | |
| CPI-360 | Histone | Epigenetics | C25H31N3O4 |
| | Methyltransferase | | |
| Picropodophyllin (PPP) | IGF-1R | Protein Tyrosine | C22H22O8 |
| | | Kinase | |
| Savolitinib(AZD6094, | c-Met | Protein Tyrosine | C17H15N9 |
| HMPL-504) | | Kinase | |
| SP2509 | Histone Demethylase | Epigenetics | C19H20ClN3O5S |
| VX-11e | ERK | MAPK | C₂₄H₂₀C₁₂FN₅O₂ |
| SBE 13 HCl | PLK | Cell Cycle | C24H28Cl2N2O4 |
| BLZ945 | CSF-1R | Protein Tyrosine | C20H22N4O3S |
| | | Kinase | |
| LFM-A13 | BTK | Angiogenesis | C11H8Br2N2O2 |
| EPZ015666(GSK3235025) | Histone | Epigenetics | C20H25N5O3 |
| | Methyltransferase | | |
| VER155008 | HSP (e.g. HSP90) | Cytoskeletal | C25H23Cl2N7O4 |
| | | Signaling | |
| BPTES | Glutaminase | Proteases | C24H24N6O2S3 |
| AZ6102 | PPAR | DNA Damage | C25H28N6O |
| Erlotinib | EGFR | Protein Tyrosine | C22H23N3O4 |
| | | Kinase | |
| ORY-1001 (RG-6016) | Histone Demethylase | Epigenetics | C15H24Cl2N2 |
| 2HCl | | | |
| EPZ020411 2HCl | Histone | Epigenetics | C25H40Cl2N4O3 |
| | Methyltransferase | | |
| I-BRD9 | Epigenetic Reader | Epigenetics | C₂₂H₂₂F₃N₃O₃S₂ |
| | Domain | | |
| SirReal2 | Sirtuin | Epigenetics | C22H20N4OS2 |
| BDA-366 | Bcl-2 | Apoptosis | C24H29N3O4 |
| NVP-CGM097 | Mdm2 | Apoptosis | C38H47ClN4O4 |
| CC-223 | mTOR | PI3K/Akt/mTOR | C21H27N5O3 |
| PFI-4 | Epigenetic Reader | Epigenetics | C21H24N4O3 |
| | Domain | | |
| BIO-acetoxime | GSK-3 | PI3K/Akt/mTOR | C18H12BrN3O3 |
| GSK2292767 | PI3K | PI3K/Akt/mTOR | C24H28N6O5S |
| SIS3 HCl | TGF-beta/Smad | TGF-beta/Smad | C28H28ClN3O3 |
| Larotrectinib (LOXO- | Trk receptor | Protein Tyrosine | C21H24F2N6O6S |
| 101) | | | |
| sulfate | | Kinase | |
| PLX7904 | Raf | MAPK | C24H22F2N6O3S |
| VPS34-IN1 | PI3K | PI3K/Akt/mTOR | C21H24ClN7O |
| A-196 | Histone | Epigenetics | C18H16Cl2N4 |
| | Methyltransferase | | |
| LDC4297 (LDC044297) | CDK | Cell Cycle | C23H28N8O |
| SMI-4a | Pim | JAK/STAT | C11H6F3NO2S |
| Empagliflozin (BI | SGLT | GPCR & G | C23H27ClO7 |
| 10773) | | | |
| | | Protein | |
| TCS 359 | FLT3 | Angiogenesis | C18H20N2O4S |
| NSC 23766 | Rho | Cell Cycle | C24H38Cl3N7 |
| GDC-0349 | mTOR | PI3K/Akt/mTOR | C24H32N6O3 |
| Cobimetinib (GDC- | MEK | MAPK | C21H21F3IN3O2 |
| 0973, | | | |
| RG7420) | | | |
| GW2580 | CSF-1R | Protein Tyrosine | C20H22N4O3 |
| | | Kinase | |
| BMS-345541 | IκB/IKK | NF-κB | C14H17N5 |
| Dynasore | Dynamin | Cytoskeletal | C18H14N2O4 |
| | | Signaling | |
| Venetoclax (ABT-199, | Bcl-2 | Apoptosis | C45H50ClN7O7S |
| GDC-0199) | | | |
| ICI-118551 | Adrenergic Receptor | GPCR & G | C17H28ClNO2 |
| Hydrochloride | | | |
| | | Protein | |
| AMG 337 | c-Met | Protein Tyrosine | C23H22FN7O3 |
| | | Kinase | |
| PF-CBP1 HCl | Epigenetic Reader | Epigenetics | C29H37ClN4O3 |
| | Domain | | |
| CPI-637 | Epigenetic Reader | Epigenetics | C22H22N6O |
| | Domain | | |
| BI-78D3 | JNK | MAPK | C13H9N5O5S2 |
| SB366791 | TRPV | Transmembrane | C16H14ClNO2 |
| | | Transporters | |
| Thiomyristoyl | Sirtuin | DNA Damage | C34H51N3O3S |
| CCT245737 | Chk | Cell Cycle | C16H16F3N7O |
| GSK6853 | Epigenetic Reader | Epigenetics | C22H27N5O3 |
| | Domain | | |
| SHP099 | phosphatase | Others | C16H21Cl4N5 |
| dihydrochloride | | | |
| Selonsertib (GS-4997) | ASK | Apoptosis | C24H24FN7O |
| KYA1797K | Wnt/bleta-catenin | Stem Cells & | C17H11KN2O6S2 |
| | | Wnt | |
| IPI-549 | PI3K | PI3K/Akt/mTOR | C30H24N8O2 |
| SGC2085 | Histone | Epigenetics | C19H24N2O2 |
| | Methyltransferase | | |
| Irbinitinib (ARRY-380, | HER2 | Protein Tyrosine | C26H24N8O2 |
| ONT-380) | | Kinase | |
| NMS-P118 | PARP | DNA Damage | C20H24F3N3O2 |
| BAY-876 | GLUT | Metabolism | C24H16F4N6O2 |
| VPS34 inhibitor 1 | PI3K | PI3K/Akt/mTOR | C21H25N7O |
| (Compound 19, PIK-III | | | |
| analogue) | | | |
| UK-371804 HCl | Serine Protease | Proteases | C₁₄H₁₇Cl₂N₅O₄S |
| GSK′872 | Serine/threonin kinase | Apoptosis | C19H17N3O2S2 |
| (GSK2399872A) | | | |
| LLY-283 | Histone | Epigenetics | C17H18N4O4 |
| | Methyltransferase | | |
| GSK180736A | ROCK | Cell Cycle | C19H16FN5O2 |
| (GSK180736) | | | |
| PD-166866 (PD166866) | FGFR | Angiogenesis | C20H24N6O3 |
| BLU-554 (BLU554) | FGFR | Angiogenesis | C24H24Cl2N4O4 |
| LY3214996 | ERK | MAPK | C22H27N7O2S |
| PF-06651600 | JAK | JAK/STAT | C15H19N5O |
| FM-381 | JAK | JAK/STAT | C24H24N6O2 |
| AZ31 | ATM/ATR | DNA Damage | C24H28N4O3 |
| Tofogliflozin(CSG 452) | SGLT | GPCR & G | C22H28O7 |
| | | Protein | |
| Omarigliptin (MK-3102) | DPP-4 | Proteases | C17H20F2N4O3S |
| Serabelisib (INK- | PI3K | PI3K/Akt/mTOR | C19H17N5O3 |
| 1117, MLN-1117, TAK- | | | |
| 117) | | | |
| FX1 | Bcl-6 | Apoptosis | C14H9ClN2O4S2 |
| Pamiparib (BGB-290) | PARP | DNA Damage | C16H15FN4O |
| NCT-503 | Dehydrogenase | Metabolism | C20H23F3N4S |
| Chk2 Inhibitor II (BML- | Chk | Cell Cycle | C20H14ClN3O2 |
| 277) | | | |
| Ipragliflozin (ASP1941) | SGLT | GPCR & G | C21H21FO5S |
| | | Protein | |
| Nec-1s (7-Cl—O-Nec1) | TNF-alpha | Apoptosis | C13H12ClN3O2 |
| GSK′963 | NF-κB, TNF-alpha | NF-κB | C14H18N2O |
| GNF-6231 | Wnt/beta-catenin | Stem Cells & | C24H25FN6O2 |
| | | Wnt | |
| Skp2 inhibitor C1 | CDK | Cell Cycle | C₁₈H₁₃BrN₂O₄S₂ |
| (SKPin | | | |
| C1) | | | |
| PF-06840003 | IDO | Metabolism | C12H9FN2O2 |
| GI254023X | Immunology & | Immunology & | C21H33N3O4 |
| | Inflammation related | Inflammation | |
| BAY 1895344 (BAY- | ATM/ATR | DNA Damage | C20H22ClN7O |
| 1895344) | | | |
| Tenalisib (RP6530) | PI3K | PI3K/Akt/mTOR | C23H18FN5O2 |
| H3B-6527 | FGFR | Protein Tyrosine | C29H34Cl2N8O4 |
| | | Kinase | |
| Cu-CPT22 | TLR | Immunology & | C19H22O7 |
| | | Inflammation | |
| AZD1390 | ATM/ATR | PI3K/Akt/mTOR | C27H32FN5O2 |
| Atuveciclib (BAY- | CDK | Cell Cycle | C18H18FN5O2S |
| 1143572) | | | |
| LXH254 | Raf | MAPK | C25H25F3N4O4 |
| WM-1119 | Histone Acetyltransf | Epigenetics | C18H13F2N3O3S |
| Evobrutinib | BTK | Protein Tyrosine | C25H27N5O2 |
| | | Kinase | |
| LIT-927 | CXCR | Immunology & | C17H13ClN2O3 |
| | | Inflammation | |
| 4-Hydroxyquinazoline | Others | antiplatelet | C8H6N2O |
| Valbenazine tosylate | VMAT2 | Others | C38H54N2O10S2 |
| SAR125844 | c-Met | Protein Tyrosine | C25H23FN8O2S2 |
| | | Kinase | |
| dBET1 | Epigenetic Reader Do | Epigenetics | C38H37ClN8O7S |
| GSK′547 | TNF-alpha | Apoptosis | C20H18F2N6O |
| Palbociclib (PD- | CDK | Cell Cycle | C24H30ClN7O2 |
| 0332991) | | | |
| HCl | | | |
| Palbociclib | CDK | Cell Cycle | C26H35N7O6S |
| (PD0332991) | | | |
| Isethionate | | | |
| LDN-193189 2HCl | TGF-beta/Smad | TGF-beta/Smad | C25H23ClN6 |
| MCC950(CP-456773) | Immunology & | Immunology & | C20H23N2NaO5S |
| | Inflammation related | Inflammation | |
| bpV (HOpic) | PTEN | Others | C6H4K2NO8V |
| Erlotinib HCl (OSI-744) | Autophagy, EGFR | Protein Tyrosine | C22H24ClN3O4 |
| | | Kinase | |
| SGX-523 | c-Met | Protein Tyrosine | C18H13N7S |
| | | Kinase | |
| (−)-Huperzine A (HupA) | GluR, AChR | Neuronal | C15H18N2O |
| | | Signaling | |
| GSK256066 | PDE | Metabolism | C27H26N4O5S |
| CCT137690 | Aurora Kinase | Cell Cycle | C26H31BrN8O |
| Capmatinib | c-Met | Protein Tyrosine | C23H17FN6O |
| (INCB28060) | | | |
| | | Kinase | |
| EPZ005687 | Histone | Epigenetics | C32H37N5O3 |
| | Methyltransferase | | |
| GSK126 | Histone | Epigenetics | C31H38N6O2 |
| | Methyltransferase | | |
| Tazemetostat (EPZ- | Histone | Epigenetics | C34H44N4O4 |
| 6438) | | | |
| | Methyltransferase | | |
| ISRIB (trans-isomer) | PERK | Apoptosis | C22H24Cl2N2O4 |
| A-1210477 | Bcl-2 | Apoptosis | C46H55N7O7S |
| Otenabant (CP-945598) | Cannabinoid Receptor | GPCR & G | C25H26Cl3N7O |
| HCl | | | |
| | | Protein | |
| FGF401 | FGFR | Protein Tyrosine | C₂₅H₃₀N₈O₄ |
| | | Kinase | |
| Lazertinib (YH25448, | EGFR | Protein Tyrosine | C₃₀H₃₄N₈O₃ |
| GNS-1480) | | Kinase | |

Aspects of the techniques described in the present disclosure may include any of the following aspects, either alone or in combination:

1. A computer-implemented quality control method for predicting whether a continuous value classifier will generate discordant classifications of a sample, comprising: receiving, via one or more processors, transcriptomic data including a plurality of observed gene expression values each corresponding to one of a plurality of samples sequenced a plurality of times; generating, via a machine learning model, a predicted standard deviation value for each of the plurality of observed gene expression values; for each sample in the plurality of samples: computing, for each simulation in a plurality of simulations, and for each of a plurality of genes, a respective simulated expression value, by drawing from a normal distribution based on an observed expression value for the gene and the predicted standard deviation value for the gene; classifying, via one or more processors, the simulated expression values using the classifier, wherein classifying the simulated expression values includes counting a number of classifications; computing, via one or more processors, a confidence score; and when the confidence score is within a predetermined range, flagging the sample as a potentially discordant sample; and storing, in an electronic database, at least one of the confidence scores in association with the transcriptomic data.

2. The computer-implemented method of aspect 1, further comprising: generating, via one or more processors, a static digital report including at least one classification result.

3. The computer-implemented method of any of aspects 1-2, wherein generating the static digital report includes censoring a no-call classification result.

4. The computer-implemented method of any of aspects 1-3, further comprising: receiving, via an application programming interface, a request for RNA sequence data; processing, via one or more processors, the request using a rules engine to determine an identity of a sender of the request; determining, via one or more processors, a role of the identity of the sender of the request; configuring, via one or more processors, a rules engine based on the role of the identity of the sender of the request; generating, via one or more processors, modified sequence data by processing the sequence data using the configured rules engine; and transmitting, via electronic network, the modified sequence data to a computing device of the sender of the request.

5. The computer-implemented method of any of aspects 1-4, wherein generating the modified sequence data by processing the sequence data using the configured rules engine includes generating censored results when the role of the identity of the sender of the request is a patient.

6. The computer-implemented method of any of aspects 1-4, wherein generating the modified sequence data by processing the sequence data using the configured rules engine includes generating annotated results when the role of the identity of the sender of the request is a clinician.

7. The computer-implemented method of any of aspects 1-4, wherein the method is performed automatically as part of a bioinformatics pipeline; wherein the role of the sender of the request is a programmatic script; and wherein generating the modified sequence data by processing the sequence data includes programmatically generating a structured output corresponding to the modified sequence data, wherein the structured output includes the modified sequence data and one or more confidence scores corresponding to the modified sequence data.

8. The computer-implemented method of any of aspects 1-7, wherein the number of classifications is two or more.

9. The computer-implemented method of any of aspects 1-8, further comprising: determining, based on flagging the sample as the potentially discordant sample, an alternative therapy that may be appropriate for a patient corresponding to the sample.

10. The computer-implemented method of any of aspects 1-9, wherein the alternative therapy includes at least one of: (i) a first-line therapy or (ii) a second-line therapy.

11. The computer-implemented method of any of aspects 1-10, wherein the alternative therapy includes at least one of: (i) a chemotherapeutic drug therapy, (ii) a radiation therapy, (iii) a surgery therapy, (iv) a therapy in a different class, (v) a molecular inhibitor therapy, (vi) an antibody therapy, (vii) a recombinant nucleic acid therapy, (viii) an engineered immune cell therapy, (ix) a checkpoint inhibitor therapy, (x) a cytokine therapy, (xi) a cancer treatment vaccine therapy, (xii) a CAR-cell therapy; or (xiii) an oncolytic virus therapy.

12. The computer-implemented method of any of aspects 1-11, wherein the machine learning model is at least one of a Bayesian model, a supervised machine learning model or an unsupervised machine learning model.

13. A computing system, comprising: one or more processors; and
one or more memories, having stored thereon instructions that when executed, cause the computing system to: receive, via the one or more processors, transcriptomic data including a plurality of observed gene expression values each corresponding to one of a plurality of samples sequenced a plurality of times; generate, via a machine learning model, a predicted standard deviation value for each of the plurality of observed gene expression values; for each sample in the plurality samples: compute, for each simulation in a plurality of simulations, and for each of a plurality of genes, a respective simulated expression value, by drawing from a normal distribution based on an observed expression value for the gene and the predicted standard deviation value for the gene; classify, via the one or more processors, the simulated expression values using the classifier, wherein classifying the simulated expression values includes counting a number of classifications; compute, via the one or more processors, a confidence score; and when the confidence score is within a predetermined range, flag the sample as a potentially discordant sample; and store, in an electronic database, at least one of the confidence scores in association with the transcriptomic data.

14. The computing system of aspect 13, the memory having stored thereon instructions that when executed, cause the computing system to: generate, via one or more processors, a static digital report including at least one classification result.

15. The computing system of any of aspects 13-14, the memory having stored thereon instructions that when executed, cause the computing system to: receive, via an application programming interface, a request for RNA sequence data; process, via one or more processors, the request using a rules engine to determine an identity of a sender of the request; determine, via one or more processors, a role of the identity of the sender of the request; configure, via one or more processors, a rules engine based on the role of the identity of the sender of the request; generate, via one or more processors, modified sequence data by processing the sequence data using the configured rules engine; and transmit, via electronic network, the modified sequence data to a computing device of the sender of the request.

16. The computing system of any of aspects 13-15, the memory having stored thereon instructions that when executed, cause the computing system to: generate censored results when a role of an identity of a sender of a request is a patient.

17. The computing system of any of aspects 13-16, the memory having stored thereon instructions that when executed, cause the computing system to: generate annotated results when a role of an identity of a sender of a request is a clinician.

18. The computing system of aspects 13-17, wherein the number of classifications is two or more.

19. The computing system of aspects 13-18, the memory having stored thereon instructions that when executed, cause the computing system to: determine, based on flagging the sample as the potentially discordant sample, an alternative therapy that may be appropriate for a patient corresponding to the sample.

20. A computer-readable media having stored thereon a set of non-transitory computer-readable instructions that, when executed, cause a computer to: receive, via one or more processors, transcriptomic data including a plurality of observed gene expression values each corresponding to one of a plurality of samples sequenced a plurality of times; generate, via a machine learning model, a predicted standard deviation value for each of the plurality of observed gene expression values; for each sample in the plurality samples: compute, for each simulation in a plurality of simulations, and for each of a plurality of genes, a respective simulated expression value, by drawing from a normal distribution based on an observed expression value for the gene and the predicted standard deviation value for the gene; classify, via one or more processors, the simulated expression values using the classifier, wherein classifying the simulated expression values includes counting a number of classifications; compute, via one or more processors, a confidence score; and when the confidence score is within a predetermined range, flag the sample as a potentially discordant sample; and store, in an electronic database, at least one of the confidence scores in association with the transcriptomic data.

### Additional Considerations

The computer-readable media may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device 1300 may represent a CPU-type processing unit, a GPU-type processing unit, a TPU-type processing unit, a field-programmable gate array (FPGA), another class of digital signal processor (DSP), or other hardware logic components that can be driven by a CPU.

A system for performing the methods described herein may include a computing device, and more particularly may be implemented on one or more processing units, for example, Central Processing Units (CPUs), and/or on one or more or Graphical Processing Units (GPUs), including clusters of CPUs and/or GPUs. Features and functions described may be stored on and implemented from one or more non-transitory computer-readable media of the computing device. The computer-readable media may include, for example, an operating system and software modules, or “engines,” that implement the methods described herein. Those engines may be stored as sets of non-transitory computer-executable instructions. The computing device may be a distributed computing system, such as an Amazon Web Services, Google Cloud Platform Microsoft Azure, or other public, private and/or hybrid cloud computing solution.

The computing device includes a network interface communicatively coupled to network, for communicating to and/or from a portable personal computer, smart phone, electronic document, tablet, and/or desktop personal computer, or other computing devices. The computing device further includes an I/O interface connected to devices, such as digital displays, user input devices, etc.

The functions of the engines may be implemented across distributed computing devices, etc. connected to one another through a communication link. In other examples, functionality of the system may be distributed across any number of devices, including the portable personal computer, smart phone, electronic document, tablet, and desktop personal computer devices shown. The computing device may be communicatively coupled to the network and another network. The networks may be public networks such as the Internet, a private network such as that of a research institution or a corporation, or any combination thereof. Networks can include, local area network (LAN), wide area network (WAN), cellular, satellite, or other network infrastructure, whether wireless or wired. The networks can utilize communications protocols, including packet-based and/or datagram-based protocols such as Internet protocol (IP), transmission control protocol (TCP), user datagram protocol (UDP), or other types of protocols. Moreover, the networks can include a number of devices that facilitate network communications and/or form a hardware basis for the networks, such as switches, routers, gateways, access points (such as a wireless access point as shown), firewalls, base stations, repeaters, backbone devices, etc.

The computer-readable media may include executable computer-readable code stored thereon for programming a computer (for example, comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device may represent a CPU-type processing unit, a GPU-type processing unit, a field-programmable gate array (FPGA), another class of digital signal processor (DSP), or other hardware logic components that can be driven by a CPU.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components or multiple components.

Additionally, certain aspects are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example aspects, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various aspects, a hardware module may be implemented mechanically or electronically. For example, a hardware module may comprise dedicated circuitry or logic that is permanently configured (e.g., as a special-purpose processor, such as a microcontroller, field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering aspects in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In aspects in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein can be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example aspects, comprise processor-implemented modules.

Similarly, the methods or routines described herein may be at least partially processor-implemented. For example, at least some of the operations of a method can be performed by one or more processors or processor-implemented hardware modules. The performance of certain of the operations may be distributed among the one or more processors, not only residing within a single machine, but also deployed across a number of machines. In some example aspects, the processor or processors may be located in a single location (e.g., within a home environment, an office environment or as a server farm), while in other aspects the processors may be distributed across a number of locations.

The performance of certain of the operations may be distributed among the one or more processors, not only residing within a single machine, but also deployed across a number of machines. In some example aspects, the one or more processors or processor-implemented modules may be located in a single geographic location (e.g., within a home environment, an office environment, or a server farm). In other example aspects, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one aspect” or “an aspect” means that a particular element, feature, structure, or characteristic described in connection with the aspect is included in at least one aspect. The appearances of the phrase “in one aspect” in various places in the specification are not necessarily all referring to the same aspect.

Some aspects may be described using the expression “coupled” and “connected” along with their derivatives. For example, some aspects may be described using the term “coupled” to indicate that two or more elements are in direct physical or electrical contact. The term “coupled,” however, may also mean that two or more elements are not in direct contact with each other, but yet still co-operate or interact with each other. The aspects are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the aspects herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible aspect, as describing every possible aspect would be impractical, if not impossible. One could implement numerous alternate aspects, using either current technology or technology developed after the filing date of this application.

What is claimed:

1. A computer-implemented quality control method for predicting whether a continuous value classifier will generate discordant classifications of a sample, comprising:
receiving, via one or more processors, transcriptomic data including a plurality of observed gene expression values each corresponding to one of a plurality of samples sequenced a plurality of times;
generating, via a machine learning model, a predicted standard deviation value for each of the plurality of observed gene expression values;
for each sample in the plurality of samples:
computing, for each simulation in a plurality of simulations, and for each of a plurality of genes, a respective simulated expression value, by drawing from a normal distribution based on an observed expression value for the gene and the predicted standard deviation value for the gene;
classifying, via one or more processors, the simulated expression values using the classifier, wherein classifying the simulated expression values includes counting a number of classifications;
computing, via one or more processors, a confidence score; and
when the confidence score is within a predetermined range, flagging the sample as a potentially discordant sample; and
storing, in an electronic database, at least one of the confidence scores in association with the transcriptomic data.

2. The computer-implemented method of claim 1, further comprising:
generating, via one or more processors, a static digital report including at least one classification result.

3. The computer-implemented method of claim 2, wherein generating the static digital report includes censoring a no-call classification result.

4. The computer-implemented method of claim 1, further comprising:
receiving, via an application programming interface, a request for RNA sequence data;
processing, via one or more processors, the request using a rules engine to determine an identity of a sender of the request;
determining, via one or more processors, a role of the identity of the sender of the request;
configuring, via one or more processors, a rules engine based on the role of the identity of the sender of the request;
generating, via one or more processors, modified sequence data by processing the sequence data using the configured rules engine; and
transmitting, via electronic network, the modified sequence data to a computing device of the sender of the request.

5. The computer-implemented method of claim 4, wherein generating the modified sequence data by processing the sequence data using the configured rules engine includes generating censored results when the role of the identity of the sender of the request is a patient.

6. The computer-implemented method of claim 4, wherein generating the modified sequence data by processing the sequence data using the configured rules engine includes generating annotated results when the role of the identity of the sender of the request is a clinician.

7. The computer-implemented method of claim 4,
wherein the method is performed automatically as part of a bioinformatics pipeline;
wherein the role of the sender of the request is a programmatic script; and
wherein generating the modified sequence data by processing the sequence data includes programmatically generating a structured output corresponding to the modified sequence data,
wherein the structured output includes the modified sequence data and one or more confidence scores corresponding to the modified sequence data.

8. The computer-implemented method of claim 1, wherein the number of classifications is two or more.

9. The computer-implemented method of claim 1, further comprising:
determining, based on flagging the sample as the potentially discordant sample, an alternative therapy that may be appropriate for a patient corresponding to the sample.

10. The computer-implemented method of claim 9, wherein the alternative therapy includes at least one of: (i) a first-line therapy or (ii) a second-line therapy.

11. The computer-implemented method of claim 9, wherein the alternative therapy includes at least one of:
(i) a chemotherapeutic drug therapy,
(ii) a radiation therapy,
(iii) a surgery therapy,
(iv) a therapy in a different class,
(v) a molecular inhibitor therapy,
(vi) an antibody therapy,
(vii) a recombinant nucleic acid therapy,
(viii) an engineered immune cell therapy,
(ix) a checkpoint inhibitor therapy,
(x) a cytokine therapy,
(xi) a cancer treatment vaccine therapy,
(xii) a CAR-cell therapy; or
(xiii) an oncolytic virus therapy.

12. The computer-implemented method of claim 1, wherein the machine learning model is at least one of a Bayesian model, a supervised machine learning model or an unsupervised machine learning model.

13. A computing system, comprising:
one or more processors; and
one or more memories, having stored thereon instructions that when executed, cause the computing system to:
receive, via the one or more processors, transcriptomic data including a plurality of observed gene expression values each corresponding to one of a plurality of samples sequenced a plurality of times;
generate, via a machine learning model, a predicted standard deviation value for each of the plurality of observed gene expression values;
for each sample in the plurality samples:
compute, for each simulation in a plurality of simulations, and for each of a plurality of genes, a respective simulated expression value, by drawing from a normal distribution based on an observed expression value for the gene and the predicted standard deviation value for the gene;
classify, via the one or more processors, the simulated expression values using the classifier, wherein classifying the simulated expression values includes counting a number of classifications;
compute, via the one or more processors, a confidence score; and
when the confidence score is within a predetermined range, flag the sample as a potentially discordant sample; and
store, in an electronic database, at least one of the confidence scores in association with the transcriptomic data.

14. The computing system of claim 13, the memory having stored thereon instructions that when executed, cause the computing system to:
generate, via one or more processors, a static digital report including at least one classification result.

15. The computing system of claim 13, the memory having stored thereon instructions that when executed, cause the computing system to:
receive, via an application programming interface, a request for RNA sequence data;
process, via one or more processors, the request using a rules engine to determine an identity of a sender of the request;
determine, via one or more processors, a role of the identity of the sender of the request;
configure, via one or more processors, a rules engine based on the role of the identity of the sender of the request;
generate, via one or more processors, modified sequence data by processing the sequence data using the configured rules engine; and
transmit, via electronic network, the modified sequence data to a computing device of the sender of the request.

16. The computing system of claim 13, the memory having stored thereon instructions that when executed, cause the computing system to:
generate censored results when a role of an identity of a sender of a request is a patient.

17. The computing system of claim 13, the memory having stored thereon instructions that when executed, cause the computing system to:
generate annotated results when a role of an identity of a sender of a request is a clinician.

18. The computing system of claim 13, wherein the number of classifications is two or more.

19. The computing system of claim 13, the memory having stored thereon instructions that when executed, cause the computing system to:
determine, based on flagging the sample as the potentially discordant sample, an alternative therapy that may be appropriate for a patient corresponding to the sample.

20. A computer-readable media having stored thereon a set of non-transitory computer-readable instructions that, when executed, cause a computer to:
receive, via one or more processors, transcriptomic data including a plurality of observed gene expression values each corresponding to one of a plurality of samples sequenced a plurality of times;
generate, via a machine learning model, a predicted standard deviation value for each of the plurality of observed gene expression values;
for each sample in the plurality samples:
compute, for each simulation in a plurality of simulations, and for each of a plurality of genes, a respective simulated expression value, by drawing from a normal distribution based on an observed expression value for the gene and the predicted standard deviation value for the gene;
classify, via one or more processors, the simulated expression values using the classifier, wherein classifying the simulated expression values includes counting a number of classifications;
compute, via one or more processors, a confidence score; and
when the confidence score is within a predetermined range, flag the sample as a potentially discordant sample; and
store, in an electronic database, at least one of the confidence scores in association with the transcriptomic data.
